(19) United States
(12) Patent Application Publication
(10) Pub. No.: US 2026/0325240 A1
(43) Pub. Date: Oct. 22, 2026

(54) ARTICULATED SURGICAL MICRO-INSTRUMENT FOR SURGICAL TELEOPERATION

(71) Applicant: MEDICAL MICROINSTRUMENTS, INC., Wilmington, DE (US)

(72) Inventors: Matteo BAGHERI GHAVIFEKR, Pisa (IT); Massimiliano SIMI, Pisa (IT)

(21) Appl. No.: 19/105,712

(22) PCT Filed: Aug. 21, 2023
(86) PCT No.: PCT/IB2023/058307

§ 371 Date: Feb. 21, 2025

(30) Foreign Application Priority Data
Aug. 25, 2022 (IT) 102022000017592

(51) Int. Cl. A61B 34/35; A61B 34/00; A61B 34/30

(52) U.S. Cl. CPC A61B 34/35; A61B 34/71; A61B 34/72; A61B 2034/305

(57) ABSTRACT

A surgical instrument includes an articulated end effector with a support structure, a first link articulated to the structure, a second link articulated to the first link; and an actuation tendon for moving the first link with respect to the support structure, The first link includes an attachment root having a winding pulley portion with a radius centered on the first rotation axis. The actuation tendon is wound around the winding pulley portion of the first link attachment root. The surgical instrument has an actuation tendon for moving the second link with respect to the first link in a closing direction of the degree of freedom of opening/closing. The second link has a circumferential actuation portion having a working radius centered on the second rotation axis. The working radius of the second link is greater than the radius of the winding pulley portion of the first link attachment root.

### CROSS-REFERENCE TO RELATED APPLICATIONS

This application is a National Stage Application of PCT/IB2023/058307, filed Aug. 21, 2023, which claims benefit of priority to Italian Patent Application No. 102022000017592 filed on Aug. 25, 2022, and which applications are incorporated herein by reference. To the extent appropriate, a claim of priority is made to each of the above disclosed applications.

### FIELD OF THE INVENTION

The present invention relates to a surgical instrument.

In particular, a surgical instrument according to the present invention comprises an articulated end effector.

A surgical instrument according to the invention is particularly suitable, although not uniquely intended, for a robotic microsurgical teleoperation system.

### PRIOR ART

Robotic surgery apparatuses are generally known in the art and typically comprise a central robotic tower (or cart) and one or more robotic arms extending from the central robotic tower. Each arm comprises a motorized positioning system (or manipulator) for moving a surgical instrument distally attachable thereto, in order to perform surgical procedures on a patient. The patient typically lies on an operating bed located in the operating room, in which sterility is ensured to avoid bacterial contamination due to non-sterile parts of the robotic apparatus.

The miniaturization of surgical instruments, and in particular of the articulated end-effectors thereof for robotic surgery, is particularly desirable because it opens up advantageous scenarios of minimal invasiveness for the patient undergoing surgery, as well as the millimeter and sub-millimeter dissection capacity of tissues.

For example, U.S. Pat. No. 10,582,975, WO-2017-064303 and WO-2018-18972 to the same Applicant disclose various embodiments of surgical instruments suitable for robotic surgery and microsurgery, where in order to miniaturize the articulations, the tendons slide and are guided in their sliding movement without the need to provide holes or concave guide channels. In contrast, the actuation tendons are supported and kept in position by suitable convex sliding surfaces, said surfaces being ruled surfaces with generatrices all parallel to one another, each ruled sliding surface being parallel to a given axis. Moreover, WO-2017-064305, EP-3362218 and EP-3597340 to the same Applicant disclose some methods for manufacturing such a type of surgical instrument.

WO-2018-189722 to the same Applicant discloses a surgical instrument in which the tendons for actuating the degree of freedom of opening/closing of the articulated end-effector, in addition to sliding on convex ruled sliding surfaces of the end-effector links, are wound on said convex ruled sliding surfaces, describing arcuate paths which subtend a particularly high winding angle. In fact, by virtue of the low sliding friction of the tendons, they are capable of remaining in contact with the convex ruled surface of a link over a relatively long and arcuate longitudinal section.

In addition, US-2021-0106393 to the same Applicant discloses some embodiments of a tendon made of intertwined polymer fibers. The use of polymer tendons allows reducing the sliding friction as compared to the use of metal tendons, and at the same time an adequate dimensioning of the tendon allows traveling winding longitudinal paths in the articulated end-effector.

Surgical instruments are also known, which are provided with articulated cutting ends, actuated by means of actuation cables wound around at least two pulleys, in which the blade holder includes a distal pulley of increased diameter with respect to a proximal pulley of the same articulated end, in an attempt to increase the cutting force by increasing the radius of the distal actuation pulley. Usually, it is desirable to maintain in any case the diameter of the blade actuation pulley within the overall size of the articulated end positioning rod or shaft, so as not to increase the transverse size of the surgical instrument. The positioning shaft typically extends in the longitudinal direction and receives the tendons or actuation cables of the articulated end therein.

The need to maximize the closing torque, and thus the closing force applied between the tips (jaws) of the articulated instrument, is felt even if the surgical instrument is not intended to perform a cutting action. For example, the surgical instrument can be required to apply a firm and durable gripping action.

In order to guide the actuation cable towards a distal actuation pulley of relatively large diameter, WO-2017-098279 employs, for example, intermediate idle guide pulleys with inclined axis which are driven in rotation by the cable itself when the distal pulley is actuated.

Articulated end-effector solutions that take advantage of the leverage effect to generate an increased closing force are also known. For example, U.S. Pat. No. 6,206,903 shows a solution in which each distal pulley is made in a separate piece with respect to the respective blade, and the blade is eccentrically associated with the pulley by a slot-pin coupling, in which the slot is eccentric with respect to the rotation axis (yaw) of the pulley. The blades are mutually articulated in a fulcrum which is placed distally to the distal pulley. By virtue of such an eccentric configuration, the relative movement between the distal pulley and the blade is forced, i.e., guided by the slot.

However, some drawbacks are apparent in such a solution, such as the fact that the transmission ratio of the closing torque is dependent on the opening/closing angle between the blades, thus resulting, when in operation, in the delivery of a closing force which is difficult to predetermine. A further drawback of this type of solution is that the eccentric mechanism imposes the advancement of the tips (jaws) during the relative clamping movement, thus determining positioning and cutting/gripping uncertainty. In addition, the operations required to make such a well-known distal articulation mechanism are unsuitable for miniaturization due to the presence of many parts to be made and assembled as well as the complexity of the suggested shapes of the pieces that which would necessarily be extremely weak, i.e., fragile. Solutions of the type disclosed above are shown, for example, in U.S. Pat. Nos. 8,137,339, 10,143,453 and 10,143,484.

A solution similar to the previous one is shown for example in WO-2022-072732, where however, both the fulcrum and the eccentric mounting slot and the pivot pin of the distal pulley are all contained within the disc-shaped volume of the distal pulley. However, such a well-known solution is unsuitable to be miniaturized while maintaining a satisfactory robustness, because it requires precision undercut machining and the assembly of various micro-parts.

Therefore, the need is strongly felt to provide a solution capable of increasing the closing and/or cutting and/or gripping torque in a miniaturized articulated surgical instrument, without imposing an increased gauge of the instrument itself, and without reducing the mobility of the articulations thereof.

In addition, the need is felt to provide a solution suitable for boosted miniaturization which is still robust and reliable when in operation, while being capable of delivering, when necessary, a closing and/or cutting torque and/or gripping torque which is repeatable and can be standardized in a mechanically advantageous manner.

### SUMMARY OF THE INVENTION

It is an object of the present invention to obviate the drawbacks complained of with reference to the prior art.

It is a further object of the present invention to provide an articulated surgical instrument adapted to be miniaturized.

According to an aspect of the invention, a surgical instrument comprises an articulated end effector comprising a support structure, a first link articulated to the support structure, and a second link articulated to the first link; in which the surgical instrument comprises a yaw actuation tendon for moving the first link with respect to the support structure, and the first link comprises an attachment root having a winding pulley portion with radius centered on the first rotation axis, and in which the yaw actuation tendon for moving the first link is wound around said winding pulley portion of the attachment root of the first link; and in which the surgical instrument further comprises a closing actuation tendon for moving the second link with respect to the first link in the closing direction of the degree of freedom of opening/closing, and the second link comprises a circumferential actuation portion having working radius centered on the second rotation axis.

According to an aspect of the invention, the working radius of the circumferential actuation portion of the second link is greater than the radius of the winding pulley portion of the attachment root of the first link.

The circumferential actuation portion of the second link can be a pulley portion having circumferential profile with working radius centered on the second rotation axis, in which the closing actuation tendon for moving the second link is wound around said pulley portion of the second link.

### BRIEF DESCRIPTION OF THE FIGURES

Further features and advantages of the invention will become apparent from the following description of preferred embodiments, given by way of non-limiting indication, with reference to the accompanying drawings which are briefly described below. It should be noted that references to “an” embodiment in this disclosure do not necessarily refer to the same embodiment, and are to be understood as at least one. Moreover, for reasons of conciseness and reduction of the total number of figures, a certain figure can be used to illustrate the features of more than one embodiment, and not all the elements of the figure can be necessary for a certain embodiment.

FIG. 1 A is a diagrammatic view showing in perspective a robotic system for surgical teleoperation, according to an embodiment.

FIG. 1B is an axonometric view of a portion of a robotic system for surgical teleoperation, according to an embodiment.

FIG. 2 A is an axonometric view of a surgical instrument, according to an embodiment.

FIG. 2 B is a diagrammatic axonometric view of a detail of an articulated end effector of a surgical instrument, according to an embodiment.

FIG. 3 is an axonometric view of an articulated end effector of a surgical instrument, according to an embodiment.

FIG. 4 A is an axonometric view of an articulated end effector of a surgical instrument, according to an embodiment.

FIGS. 4 B and 4 C are axonometric views from different points of view of a portion of the articulated end effector in FIG. 4 A.

FIG. 4 D shows the articulated end effector in FIG. 4 A in a different operating configuration.

FIG. 4 E shows the articulated end effector in FIG. 4 A in separate parts.

FIG. 5 is a diagrammatic plan view of an articulated end effector, according to an embodiment, in which some parts are transparent for clarity.

FIG. 6 is a diagrammatic plan view diagrammatically showing a second link, according to an embodiment.

FIGS. 7 A and 7 B are diagrammatic plan views of a portion of an articulated end effector, according to an embodiment, in closed and open configuration, respectively, in which an actuation tendon is diagrammatically shown.

FIGS. 8 A, 8 B and 8 C are diagrammatic plan views of a portion of an articulated end effector, according to an embodiment, illustrated in open, closed and maximally open configuration, respectively.

FIG. 9 is a plan view of an articulated end effector, according to an embodiment.

FIG. 10 A is a plan view of a second link, according to an embodiment.

FIG. 10 B is a plan view according to the point of view indicated by arrow B in FIG. 10 A.

FIG. 11 A is a plan view of a first link, according to an embodiment.

FIG. 11 B is a plan view according to the point of view indicated by arrow B in FIG. 11 A.

FIG. 12 is an axonometric view of the second link in FIG. 10.

FIG. 13 is an axonometric view of the first link in FIG. 11.

FIG. 14 A is an axonometric view of an articulated end effector, according to an embodiment.

FIGS. 14 B and 14 C are axonometric and plan views, respectively, of the articulated end effector in FIG. 14 A, in which some parts are in phantom for clarity.

FIGS. 15 A and 15 B diagrammatically show an axonometric view of some possible steps of a manufacturing method, according to a possible operating mode.

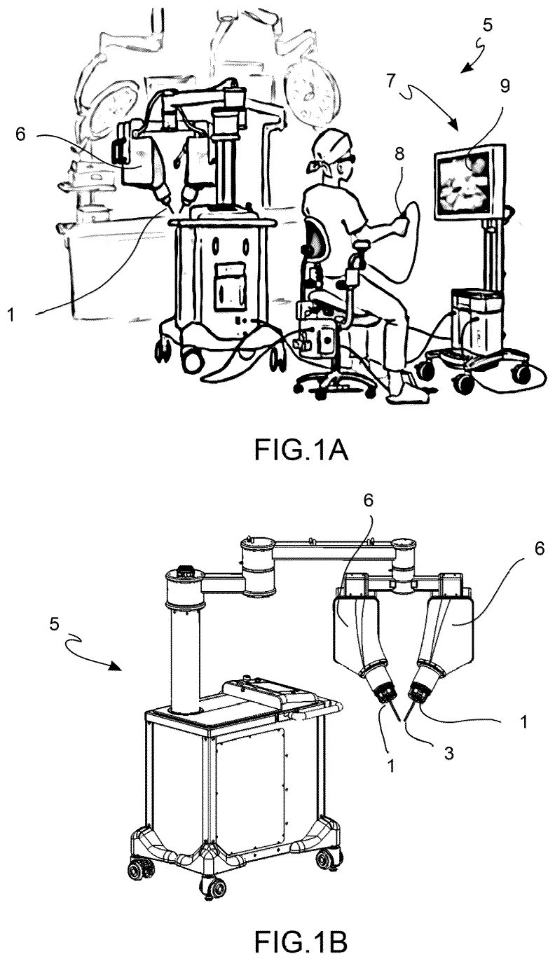

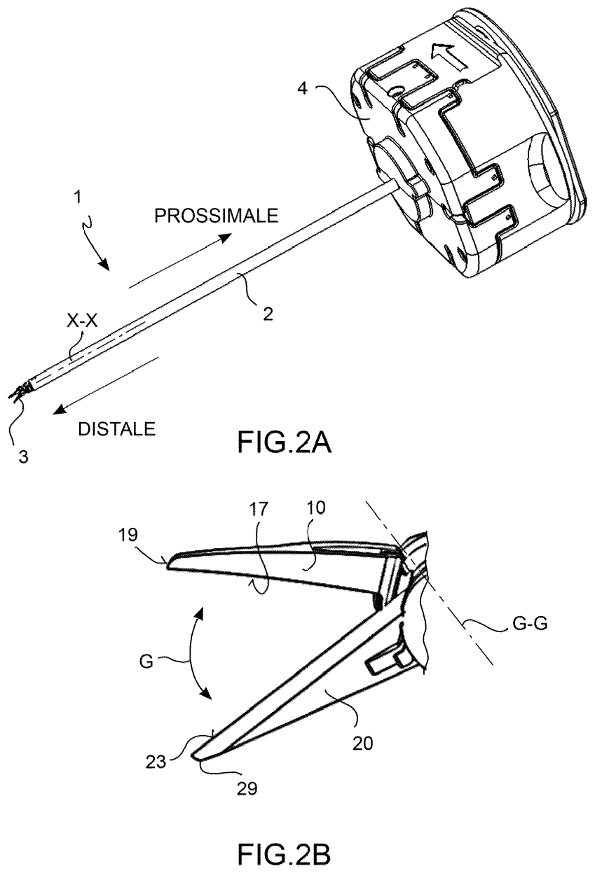

### DETAILED DESCRIPTION OF THE INVENTION

Reference throughout this description to “an embodiment” means that a particular feature, structure or function described in relation to the embodiment is included in at least one embodiment of the present invention. Therefore, the formulation “in an embodiment” in various parts of this description do not necessarily all refer to the same embodiment. Moreover, particular features, structures or functions such as those shown in different drawings can be combined in any suitable manner in one or more embodiments.

In accordance with a general embodiment, a surgical instrument 1 is provided, comprising an articulated end effector device 3 (or “articulated end-effector 3” or even just “articulated end 3”) having at least one degree of freedom of opening/closing G (also referred to as “grip” G or “cut” G).

The articulated end effector device 3 comprises a support structure, and a first link 10 articulated to the support structure so that the first link 10 and the support structure can rotate relatively about a first rotation axis Y-Y. The support structure can be an articulated structure, comprising one or more links 43, 50.

The articulated end effector device 3 further comprises a second link 20 articulated to the first link so that the second link 20 and the first link 10 can rotate relatively about a second rotation axis G-G jointly defining a degree of freedom of opening/closing G of the articulated end effector 3.

The surgical instrument comprises an actuation tendon 33 or 34, and preferably a pair of actuation tendons 33, 34 with antagonistic effects for moving the first link 10 with respect to the support structure. Preferably the antagonistic actuation tendons 33, 34 are fixed to the first link 10.

The first link comprises an attachment root 13 having a winding pulley portion 18 with radius R1 centered on the first rotation axis Y-Y, and the actuation tendon 33 or 34 for moving the first link is wound around said winding pulley portion 18 of the attachment root 13 of the first link 10. Preferably both antagonistic actuation tendons 33, 34 are fixed to the first link and wound around the winding pulley portion 18 of the attachment root 13, in circumferentially opposite directions.

The surgical instrument further comprises an actuation tendon 32 for moving the second link 20 with respect to the first link 10 in the closing direction of the degree of freedom of opening/closing G. Preferably, the surgical instrument 1 comprises two actuation tendons 31, 32 for moving the second link 20 with respect to the first link 10 with antagonistic effects. The actuation tendon 31 is thus configured to move the second link 20 in the opening direction of the degree of freedom of opening/closing G.

The second link 20 comprises a circumferential actuation portion having working radius R2 centered on the second rotation axis G-G.

Not necessarily the actuation tendon 32 and/or the actuation tendon 31 for moving the second link 20 is fixed to the second link 20.

In accordance with an embodiment, the actuation tendon 32 is fixed to the second link 20 and is wound around the circumferential actuation portion, which in such a case can be a pulley portion 28, of the second link 20 having a working radius R2 equal to the radius of the circumferential profile of the pulley portion 28.

Advantageously, the working radius R2 of the circumferential actuation portion of the second link 20 is greater than the radius R1 of the winding pulley portion 18 of the attachment root 13 of the first link 10.

The first rotation axis Y-Y and the second rotation axis G-G are separated and disjoined from each other. According to an embodiment, the first rotation axis Y-Y and the second rotation axis G-G are parallel to each other and longitudinally spaced by a longitudinal distance X1.

In accordance with a preferred embodiment, the second link 20 comprises a body having a proximal tail 25, extending proximally to the second rotation axis G-G, and in which said proximal tail 25 of the second link 20 comprises said pulley portion 28 with circumferential profile.

The articulated end effector 3 can comprise a sliding hub 40 arranged proximally to the second link 20, and in which the actuation tendon 32 is wound around the sliding hub 40 in longitudinal sliding contact. Preferably, the sliding hub 40 comprises at least one convex sliding surface 45, which is a convex ruled surface with straight generators all parallel to each other. For example, such an at least one convex sliding surface 45 of the sliding hub 40 is a cylindrical surface.

In accordance with another embodiment, the circumferential actuation portion of the second link 20 comprises a toothed circumferential profile 142 having a working radius R2, and the articulated end effector comprises a toothed link 140 mounted on the rotation axis Y-Y comprises a toothed circumferential counter-portion 141 centered on the first rotation axis Y-Y and operatively coupled to the toothed portion 142 of the toothed link 140. In such a case, the toothed link 140 further comprises a pulley portion 118 having a radius centered on the rotation axis Y-Y, the actuation tendon 32 being wound around the pulley portion 118 of the toothed link 140.

Preferably, the working radius R2 of the toothed circumferential profile 142 of the second link 20 is greater than the radius of the pulley portion 118 of the toothed link 140. If said toothed circumferential profile 142 is provided, the working radius R2 can be the transmission (or “primitive”) radius.

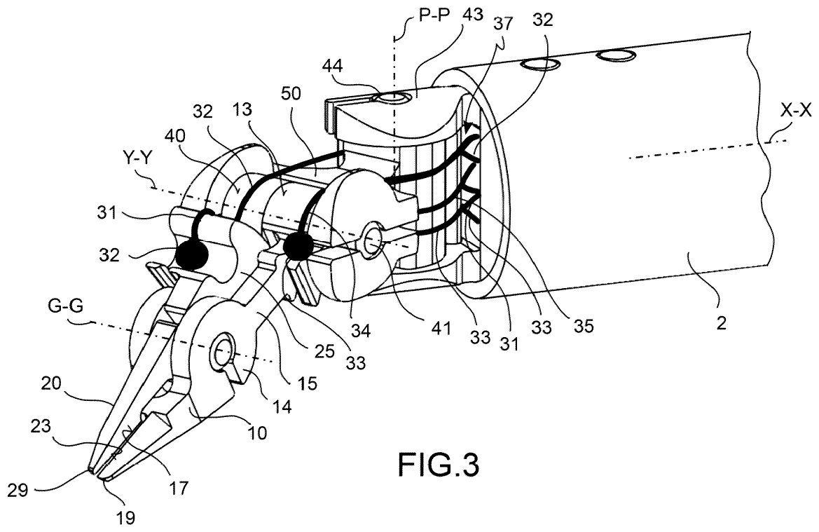

As shown for example in FIG. 3, the surgical instrument 1 can further comprise a positioning shaft 2, and the articulated end effector 3 is articulated with respect to said positioning shaft 2. For example, the articulated end effector 3 comprises a degree of freedom of opening/closing G and at least one degree of freedom of pitch P and/or yaw Y orientation with respect to the positioning shaft 2. The positioning shaft preferably extends in the longitudinal direction X-X defining a longitudinal direction of the surgical instrument 1; at least one configuration of the articulated end effector 3 substantially aligned with the longitudinal direction X-X can also be defined: this at least one configuration aligned with the longitudinal direction can correspond to a configuration with minimum transverse size.

The first link 10 is articulated with respect to the positioning shaft 2. In other words, the first link 10 has at least one degree of freedom of orientation with respect to the positioning shaft 2. The first link 10 is not necessarily directly articulated to the positioning shaft 2, and for example a support structure (for example a support link 50 and a proximal link 43 articulated about a pitch rotation axis P-P by means of a pivot pin 44, forming a pitch articulation P) can be interposed between the distal end of the positioning shaft 2 and the first link 10. The support structure can be integral with the positioning shaft 2.

The proximal attachment root 13 of the first link 10 can cooperate with a pivot pin 41 to define said first rotation axis Y-Y. For example said first rotation axis Y-Y is a rotation axis of a yaw articulation of the articulated end effector 3.

The terms pitch P and yaw Y articulation are used here arbitrarily, for clarity of exposition, and the rotation axes Y-Y and P-P mentioned can have any relative orientation (they can also be parallel to each other), although in accordance with a preferred embodiment, the yaw rotation axis Y-Y is orthogonal to the pitch rotation axis P-P.

Preferably, the first link 10 comprises a first connecting portion 14 thereof, and the second link 20 comprises a second connecting portion 24 thereof, the first connecting portion 14 of the first link 10 and the second connecting portion 24 of the second link 20 jointly define a second rotation axis G-G for the relative rotation between the second link 20 and the first link 10. The degree of freedom of opening/closing G of the articulated end effector 3 is thus achieved. In other words, the second link 20 is directly articulated to the first link 10, by means of the provision of said respective connecting portions 14, 24. Preferably, a second pivot pin 42 is provided, which articulates said second connecting portion 24 of the second link 20 with respect to said first connecting portion 14 of the first link 10.

By virtue of the fact that the first rotation axis Y-Y and the second rotation axis G-G are separated and disjoined from each other. The yaw articulation Y is thus decoupled from the opening/closing (grip) articulation G.

When in operation, the second link 20 can be driven in rotation with respect to the rotation axis Y-Y by the first link 10, when the yaw degree of freedom Y is activated.

When in operation, the second link 20 can move with respect to the first link 10 when the degree of freedom of opening/closing G is activated.

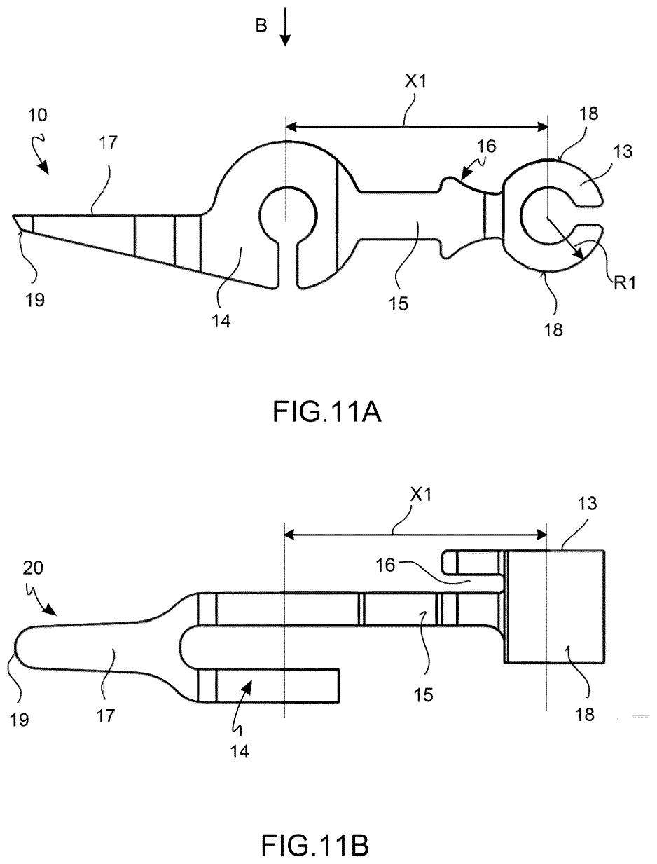

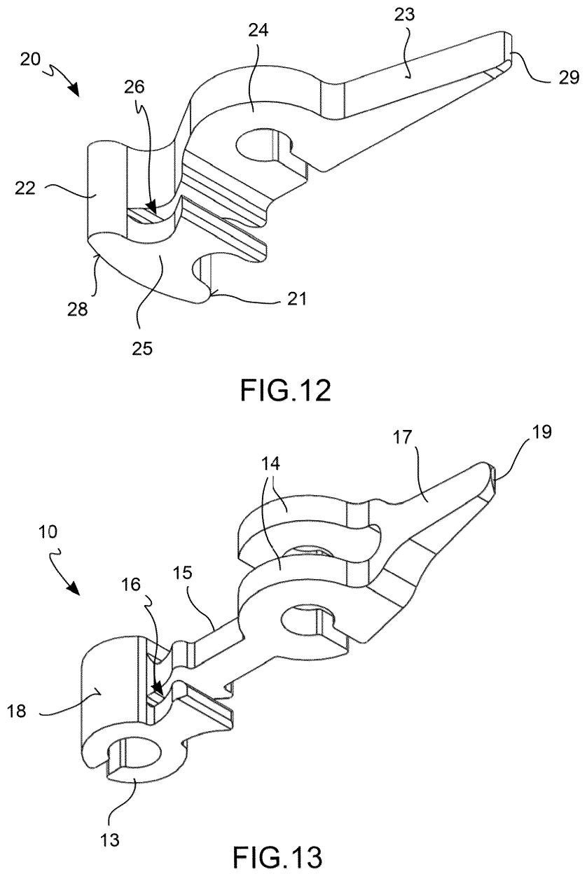

The body of the first link 10 preferably comprises a proximal section 15 or proximal portion 15 extending between the attachment root 13 forming the rotation axis Y-Y and the connecting portion 14 forming the rotation axis G-G. As shown for example in FIG. 11a, the attachment root 13 can have a substantially cylindrical shape to contour the rotation axis Y-Y, and preferably a through hole is provided to receive the pivot pin 41. The connecting portion 14 of the first link 10 can also have a shape which surrounds the respective rotation axis G-G, and according to an embodiment shown for example in FIG. 11 B, is formed by a clevis portion which embraces the respective connecting portion 24 of the second link 20. In other words, in accordance with this embodiment, the connecting portion 14 is formed by two longitudinally adjacent arms, like a fork.

In accordance with an embodiment, the articulated end effector 3 further comprises a support link 50 which is articulated with respect to at least said first link 10 about said first rotation axis Y-Y. The support link 50 can carry out a function of supporting the support structure for the first link 10 and to this end can comprise at least one prong 51, and preferably a pair of prongs 51, which mount the pivot pin 41, so that the first link 10 is articulated with respect to the prongs 41 of the link 50. At least one further proximal link 43 can be provided proximally to said support link 50, which is articulated with respect to said support link 50 about a rotation axis P-P. The proximal link 43 can be fixed to the positioning shaft 2 (e.g., by double-pin fastening).

In accordance with a preferred embodiment, the support link 50 defines, in a single piece, two rotation axes Y-Y and P-P orthogonal to each other.

The links of the surgical instrument 1 can all be manufactured by means of a wire electro-erosion process which includes making two cuts with cutting wire on planes orthogonal to each other.

Such a surgical instrument 1 is adapted to be mounted on a robotic system 5 for medical and/or surgical and/or microsurgical teleoperation. The robotic system 5 can comprise a master console 7 for controlling one or more robotic manipulators 6. The master console 7 can in turn comprise one or more master control devices 8 as well as a screen 9 for displaying the surgical site where the articulated end effector 3 operates.

Preferably, the surgical instrument 1 comprises at the proximal end of the positioning shaft 2 a transmission portion 4 (or “back-end 4”) which forms a proximal interface for interfacing with a mutual actuation portion of the robotic manipulator 6. For example, the transmission portion 4 can comprise a set of transmission elements adapted to be moved through a sterile barrier by a respective set of actuation elements of the robotic manipulator 6.

As mentioned above, in accordance with an embodiment, the surgical instrument 1 comprises a pair of antagonistic actuation tendons 31, 32 for moving the second link 20 with respect to the first link 10. Thereby, the antagonistic tendons 31, 32 of said pair are the actuation tendons, with antagonistic effect, of the degree of freedom of opening/closing G of the articulated end effector 3. For example, the antagonistic tendons 31, 32 extend from the articulated end effector 3 along the positioning shaft 2 to the transmission portion 4, where they are configured in operating connection with respective antagonistic transmission elements, which can in turn be arranged for the operating connection, when in operation, with respective antagonistic actuation elements of the robotic manipulator 6.

In accordance with an embodiment, at least one actuation tendon of the pair of antagonistic actuation tendons 31, 32 is fastened to the second link 20. In accordance with a preferred embodiment, both antagonistic actuation tendons 31, 32 are fastened to the second link 20. In other words, the actuation tendons 31, 32 of the second link 20, i.e., of the degree of freedom of opening/closing G, are fastened to the body of the second link 20 itself.

In accordance with an embodiment, the body of the second link 20 comprises a distal operating portion 23 extending distally to the connecting portion 24, and a proximal actuation tail 25 extending proximally to the connecting portion 24. In other words, the body of the second link 20 comprises two opposite portions 23, 25 extending substantially in opposite directions with respect to the rotation axis G-G.

Therefore, the body of the second link 20 extends both proximally and distally to the rotation axis G-G. Therefore, when in operation, both the distal operating portion 23 and the proximal transmission tail 25 of the second link 20 tilt, i.e., rotate, with respect to the rotation axis G-G, so that when the degree of freedom of opening/closing G is closed, and the operating portion 23 of the second link 20 abuts and/or contacts the operating portion 17 of the first link 10, the proximal tail 25 of the second link 20 is oriented in a certain direction which is different from the orientation thereof when the degree of freedom of opening/closing G is in opening condition, for example maximum opening i.e., maximum angular distance between the operating portion 23 of the second link 20 and the operating portion 17 of the first link 10.

The distal operating portion 23 and the proximal actuation tail 25 of the second link 20 do not necessarily both extend along a same straight line, for example along a definable centerline of the second link 20, and can for example both extend substantially radially, i.e., along a radial direction with respect to the rotation axis G-G, forming an angle β therebetween which is definable as respective longitudinal extension directions 123, 125. For example, the angle β is chosen such as to minimize the transverse size of the proximal tail 25 during the radial operating stroke thereof.

By virtue of this expedient, it is possible to optimize the transverse size taken by the tail 25 of the second link during the movement thereof when in operation. For example, the angle β between the two extension directions of the body of the second link 20 can be chosen so that the longitudinal extension direction 125 of the proximal transmission tail 25 is substantially aligned with a proximal stretch 15 of the first link 10, when the degree of freedom of opening/closing G is in an open configuration. Alternatively or additionally, the angle β can be chosen so that, when the degree of freedom of opening/closing G is in a closed configuration, the proximal tail 25 is misaligned i.e., protruding with respect to the proximal section 15 of the first link 10.

In accordance with an embodiment, the angle β is chosen so that the proximal tail 25 of the second link is transversely protruding, i.e., misaligned, with respect to the proximal section 15 of the first link 10, in transversely opposite directions, both when the degree of freedom of opening/closing G is in the closed configuration, and when the degree of freedom of opening/closing G is in the maximally open configuration within the limits of a definable maximum working opening angle of the degree of freedom of opening/closing G. In other words, in accordance with this embodiment, the extension of the proximal tail 25 of the second link 20 with respect to the distal operating portion 23 of the same second link 20 is chosen so that the proximal tail 25 travels a circumferential trajectory about the rotation axis G-G so that a portion of the proximal tail 25 protrudes transversely from a first side with respect to the proximal portion 15 of the first link when the links 10, 20 are at the closing end-of-stroke and protrudes transversely from an opposite second side of the same proximal portion 15 of the first link 10 when the link 20 is in a maximally open configuration with respect to the first link 10.

In a closed configuration of the degree of freedom of opening/closing G, the longitudinal extension direction 123 of the operating distal portion 23 of the second link 20 can extend aligned, i.e., substantially parallel, to a definable longitudinal extension direction of the proximal section 15 of the first link 10.

The body of the second link 20 is preferably made in a single piece.

The distal operating portion 23 of the second link 20 can comprise a blade, as shown for example in FIG. 3. The distal operating portion 23 of the second link 20 preferably comprises a distal free end 29 forming a distal end of the articulated end effector 3. The first link 10 can comprise a distal end 19 as well, which together with the distal end 29 of the second link 20 forms a distal end of the articulated end effector 3 of the surgical instrument 1. The first link 10 can comprise an operating portion 17 extending distally to the connecting portion 14 and which is designed to cooperate with the operating portion 23 of the second link 20. For example, the operating portion 17 of the first link 10 can comprise a blade or a counter-blade.

In accordance with a preferred embodiment, at least one actuation tendon 32 of the pair of antagonistic actuation tendons 31, 32 is wound around the proximal tail 25 of the second link 20, so as to actuate the second link 20 in a closing direction of the degree of freedom of opening/closing G. Such an actuation tendon 32 is preferably wound around a pulley portion 28 of the proximal actuation tail 25 of the second link 20, and ends at a termination seat 26 also provided in the proximal tail 25 of the second link 20. In other words, the actuation tendon 32 is preferably wound around and fixed to the proximal tail 25 of the second link 20.

By virtue of the joint provision of said pulley portion 28 provided on the proximal tail 25 of the second link and the longitudinal decoupling of the rotation axes Y-Y and G-G, it is possible to utilize the distance X1 between said rotation axes of yaw Y-Y and opening/closing G-G to increase the working radius of the pulley portion 28, thus increasing the closing (and/or opening) torque of the degree of freedom of opening/closing G, with the same pulling force applied by the actuation tendon 32.

The distance X1 between the rotation axes G-G and Y-Y can be chosen to obtain the desired transmission ratio of the closing force, since the working radius R2 of the second link 20 is contained in such a distance X1, i.e., in other words, increasing the distance X1 between the axes during the design stage allows the closing force to be adjusted because the tail 25 of the second link carrying such a working radius R2 can be lengthened.

An increased closing force is also desirable because it can result in an increased cutting force, or in addition in an increased clamping force, with the same pulling force applied by the actuation tendon 32.

The actuation tendons 31, 32, 33, 34, 35, 36 can be tendons formed by intertwined polymer fibers.

The pulley portion 28 of the second link 20 preferably has a circumferential profile corresponding to a section of a pulley, i.e., a circumferential chord AB, having center in the rotation axis G-G. It is thus possible to take advantage of the longitudinal distance X1 between the rotation axes Y-Y and G-G to make a pulley portion 28 with circumferential profile having a greater radius R2 than the transverse extension of the articulated end effector 3, thus increasing the lever arm of the closing torque of the articulated end effector 3, without increasing the transverse volume of the articulated end effector 3 itself.

The pulley portion 28 with circumferential profile centered on the rotation axis G-G allows to deliver a closing torque which is constant.

The pulley portion 28 can extend substantially symmetrically with respect to the longitudinal extension direction 125 of the proximal tail 25 of the second link 20, i.e., the longitudinal extension direction 125 of the proximal tail 25 is a radius which passes substantially in the center of the chord described by the circumferential profile of the pulley portion 28.

The proximal tail 25 of the second link 20 preferably comprises a return surface 22 placed at a radial margin of the pulley portion 28, in which the actuation tendon 32 winds around the pulley portion 28 and the return surface 22, then ending at the termination seat 26 thereof which is provided in the proximal tail 25. The distal end 38 of the actuation tendon 32 can comprise an enlarged portion which cooperates with driving walls of the termination seat 26 placed as an undercut to drive in rotation the second link 20 with respect to the first link 10 in the closing direction of the degree of freedom of opening/closing G. Preferably, the curvature of the return surface 22 is more accentuated than the curvature of the pulley portion 28, to obtain a return on the actuation tendon 32.

By virtue of such a configuration of the actuation tendon 32, it is possible to utilize the entire working radius R2 of the pulley portion 28 (i.e., the geometric radius of the circumferential profile thereof) to maximize the lever arm for the actuation of the degree of freedom of opening/closing G in the closing direction.

The termination seat 26 is preferably a seat circumferentially passing through the body of the proximal tail 25 of the second link 20. For example, a single through seat can obtain the termination seat 26 for both antagonistic actuation tendons 31, 32 of said pair, in which the antagonistic actuation tendons 31 and 32 are oriented in circumferentially opposite directions in the termination seat. Preferably, the termination seat 26 is placed at a lower radial height with radial the radius R2 of the pulley portion 28 and the return surface 22 which receive the winding of the actuation tendon 32. In other words, the winding pulley portion 28 for the actuation tendon 32 of the proximal actuation tail 25 of the second link 20 is further from the rotation axis G-G than the termination seat 26 for the same actuation tendon 32. It is thus possible to house the termination seat 26 in the transverse and/or radial volume of the pulley portion 28 of the proximal tail 25 of the body of the second link 20.

As mentioned above, the other actuation tendon 31 of the pair of antagonistic tendons 31, 32 can also be wound around the proximal transmission tail 25 of the second link 20, and preferably is wound around the same pulley portion 28 with a circumferential profile, for moving the second link 20 with respect to the first link 10 along the degree of freedom of opening/closing G in the opening direction. In accordance with an embodiment, the proximal tail 25 further comprises another return surface 21 at the opposite radial margin of the pulley portion 28 with respect to the return surface 22. Said other return surface 21 can be analogous to the return surface 22. The actuation tendon 31 can also terminate in a termination seat 26 which can be analogous to, and the same as, the termination seat 26 described with reference to the actuation tendon 32.

The transverse dimension Y2 of the proximal tail 25 of the second link 20 is preferably maximum at the pulley portion 28. In other words, the transverse dimension Y2 of the proximal tail 25 is maximum between the two opposite return surfaces 21, 22 (radially opposite to the pulley portion 28). Close to or at the termination seats 26, the body of the proximal tail 25 of the second link 20 can comprise a transverse groove, which locally reduces the transverse extension of the proximal tail 25 of the second link 20.

In accordance with an embodiment, the transverse dimension Y2 of the proximal tail 25 is smaller, and preferably much smaller, than the longitudinal extension thereof, i.e., the radius of the portion and pulley 28. The proximal tail 25 thus has a substantially elongated shape to minimize the transverse volume while maximizing the working radius R2 of the pulley portion 28 and thus the lever arm of the closing torque of the articulated end effector.

In accordance with a preferred embodiment, the working radius R2 of the pulley portion 28 is greater than the radius of the positioning shaft 2 and preferably greater than the diameter of the positioning shaft 2 of the surgical instrument 1, while the transverse dimension Y2 of the proximal tail 25 of the second link 20 is smaller than the diameter of the positioning shaft.

The antagonistic actuation tendons 31, 32 of the pair of actuation tendons for moving the second link 20 with respect to the first link 10 can also be wound around the body of a further link 40 (also referred to as the “sliding hub 40” herein) arranged proximally to the second link 20. In accordance with a preferred embodiment, the articulated end effector 3 comprises a further link 40 on which both antagonistic actuation tendons 31, 32 for moving the second link 20 wind and slide, when in operation; therefore, said further link 40 acts as a sliding hub 40 for the antagonistic actuation tendons 31, 32 of the pair of antagonistic tendons for moving the second link 20.

The sliding hub 40 is preferably mounted on the pivot pin 41 alongside the attachment root 13 of the first link 10, and for example between the prongs 51 of the support link 50. The sliding hub 40 can be keyed onto the pivot pin 41. In accordance with an embodiment, the sliding hub 40 is formed by the pivot pin 41 itself, and therefore in accordance with this embodiment, the further link 40 is not provided because the sliding of the actuation tendons 31, 32 occurs on the body of the pivot pin 41 itself.

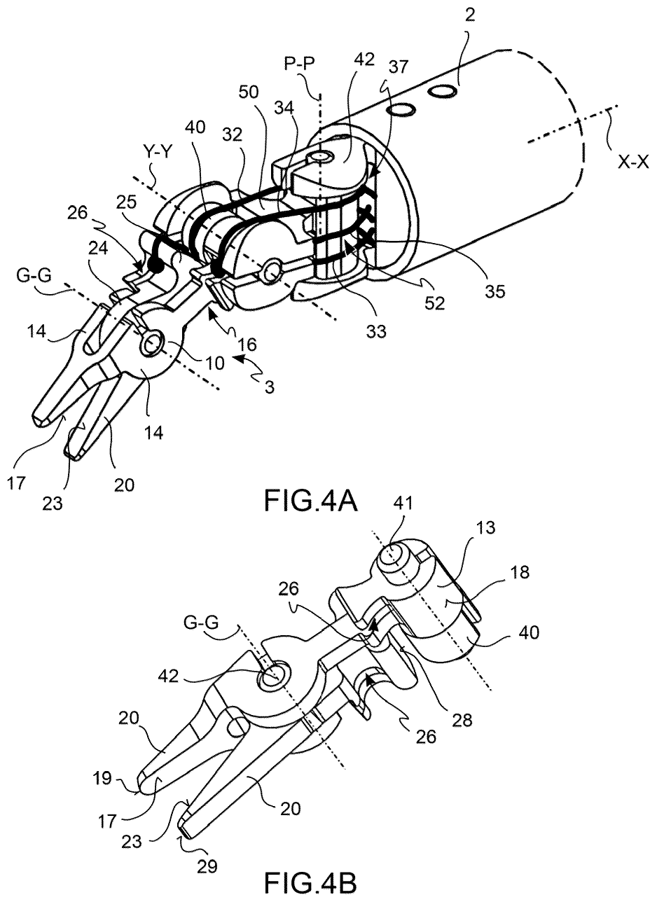

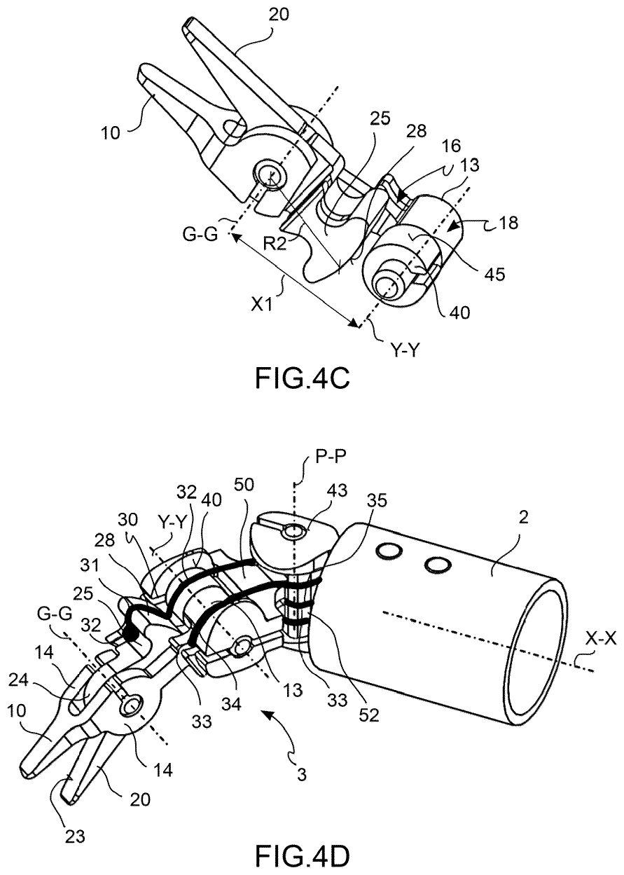

As shown for example in FIG. 4E, the body of the sliding hub 40 can have a substantially cylindrical shape around the pivot pin 41 of the yaw rotation axis Y-Y, in which the diameter of the cylinder is chosen so as to be aligned with the sliding track of the actuation tendons 31, 32 on the links 50, 43 of the articulated end effector 3, where provided. In accordance with a preferred embodiment, the radius of the sliding hub 40 is substantially equal to the radius of the winding pulley 18 of the attachment root 13 of the first link 18.

For example, in fact, as mentioned above, where the articulated end effector 3 also comprises said support link 50, the antagonistic actuation tendons 31, 32 for moving the second link 20 can slide on at least one sliding surface 52 provided on the body of the support link 50. Preferably, said at least one sliding surface 52 of the link 50 is a convex ruled surface with straight generators all parallel to each other, and preferably all parallel to the pitch rotation axis P-P of the link 50.

Where the articulated end effector 3 further comprises a still further proximal link 42, the antagonistic actuation tendons 31, 32 for moving the second link 20 can also slide on at least one sliding surface 53 provided on the body of the proximal link 42.

Preferably, the first link 10 is moved about the yaw rotation axis Y-Y by providing a further pair of antagonistic actuation tendons 33, 34. Preferably, said antagonistic actuation tendons 33, 34 of the pair are analogous to the antagonistic actuation tendons 31, 32. The body of the first link 10 preferably comprises at least one termination seat 16, and the antagonistic actuation tendons 33, 34 are fastened to said at least one termination seat 16.

In accordance with a preferred embodiment, said termination seat 16 is adjacent to the attachment root 13 of the first link 10 and placed distally with respect thereto. Both termination seats 16 and 26, of degrees of freedom of yaw Y and opening/closing G, respectively, are thus located between the first and second rotation axes Y-Y and G-G. The antagonistic actuation tendons 33, 34 are preferably wound around the attachment root 13 of the first link 10 and are terminated in the at least one termination seat 16. Both antagonistic actuation tendons 33, 34 for moving the first link 10 can slide, when in operation, on said at least one sliding surface 52 of the link 50, where provided, as well as on said at least one sliding surface 53 of the link 42, where included.

As mentioned above, in accordance with a preferred embodiment, the antagonistic actuation tendons 31, 32 for moving the second link 20 about the rotation axis G-G form a cross 30 with each other between the rotation axes G-G and Y-Y. Preferably, where a support link 50 articulated about a further proximal rotation axis P-P is provided, the same antagonistic tendons 31, 32 form a further proximal cross 37, upstream of the proximal rotation axis P-P, with respective antagonistic tendons 33, 34 of the pair of antagonistic tendons for moving the first link 10 about the rotation axis Y-Y. In other words, in accordance with this embodiment as shown for example in FIG. 4A, the closing tendon 32 (second link 20) forms a proximal cross 37, upstream of the pitch rotation axis P-P, with the tendon 34 for moving the first link 10, and forms a cross 30 between the rotation axes Y-Y and G-G with the antagonistic tendon 31 (opening tendon 31) thereof. In addition, in accordance with this embodiment, the opening tendon 31 forms a proximal cross 37 with the tendon 33 for moving the first link 10 (antagonistic tendon of tendon 34).

In accordance with an embodiment, the proximal rotation axis P-P (pitch) is orthogonal to the rotation axis Y-Y (yaw) and the rotation axis G-G, and in which the crosses 30 and 37 between the actuation tendons 31, 32, 33, 34 occur on two orthogonal planes.

Where said further proximal link 43 is provided, it is possible that the link 50 is movable by a further pair of antagonistic actuation tendons 35, 36.

Therefore, in accordance with a preferred embodiment, the winding hub 40 lacks any termination seats because it is not actuated by any actuation tendon, and acts as a return element in sliding contact with a section of each transmission tendon of the pair of antagonistic transmission tendons 31, 32 for the second link 20. For example, by virtue of said sliding hub 40, it is possible to maintain a sliding abutment aligned with the sliding path which the respective tendon 31, 32 describes on the at least one convex ruled sliding surface 52 of the link 50, as well as of the further proximal link 43, where provided. It is therefore possible to avoid the presence of further sliding guide abutments for the tendons 31, 32, without thereby imposing design limitations on the transverse dimension Y2 of the winding pulley 25 due to guide or return needs of the actuation tendons 31, 32 of said pair.

In accordance with a preferred embodiment, between the sliding hub 40 of the articulated end effector 3 (i.e., between the yaw rotation axis Y-Y) and the pulley portion 28 of the second link 20, the path of the actuation tendon 31 of the second link 20 crosses with the path of the actuation tendon 32 of the second link 20, forming a cross 30. The term “cross 30” is not necessarily intended to indicate a crossing point with sliding contact between tendon 31 and tendon 32, and the cross 30 can also occur without contact between the tendons 31, 32, as a projection on a longitudinal plane transverse to the articulated end effector 3.

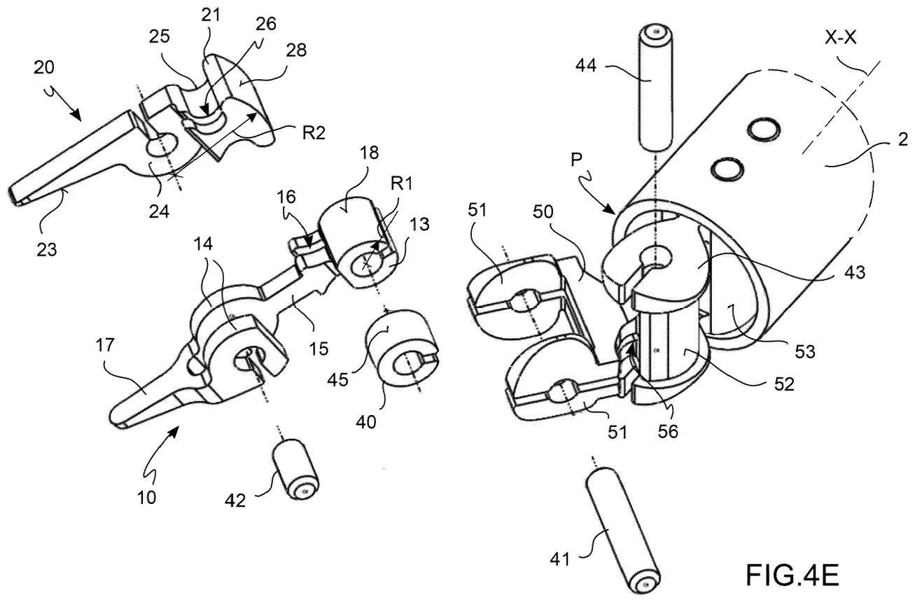

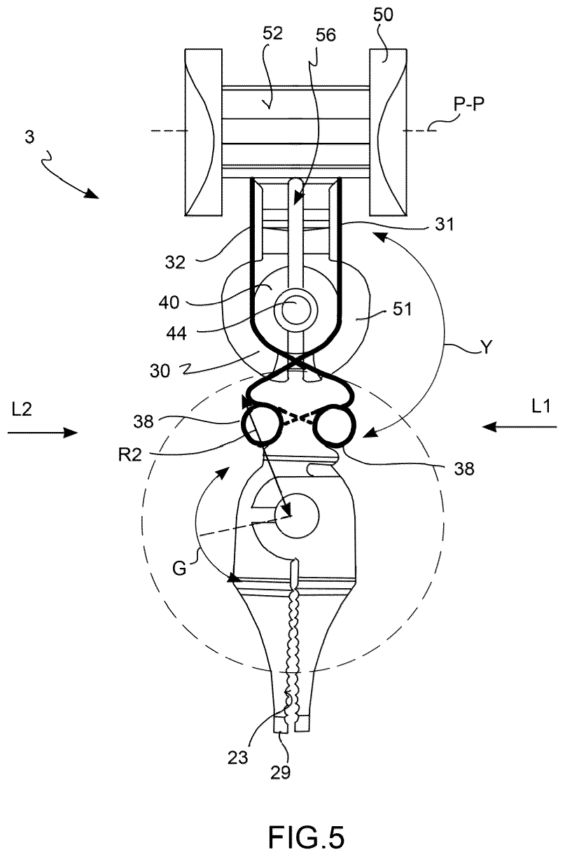

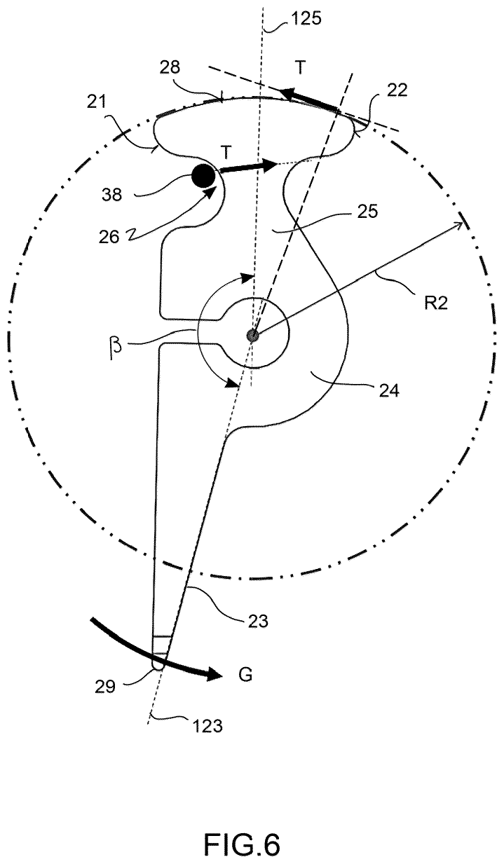

As shown for example in FIG. 5, the actuation tendon 32 is wound with longitudinal sliding contact on one side L2 of the sliding hub 40 and then continuing in the distal direction it is wound in the opposite direction on the pulley portion 28 of the second link 20 on the side L1 where the return surface 22 is provided, which side is transversely opposite to the winding side L2 of the sliding hub 40. It should be noted that the contact of the actuation tendon 32 on the pulley portion 28 does not include the relative longitudinal sliding, unlike the contact of the same actuation tendon 31 with the winding hub 40. Similarly, the actuation tendon 31 is wound with longitudinal sliding contact on one side L1 of the sliding hub 40 and then continuing in the distal direction it is wound in the opposite direction on the pulley portion 28 of the second link 20 on the side L2 where the return surface 21 is provided, which side is transversely opposite to the winding side L1 of the sliding hub 40. It should be noted that the contact of the actuation tendon 31 on the pulley portion 28 does not include the relative longitudinal sliding, unlike the contact of the same actuation tendon 31 with the winding hub 40.

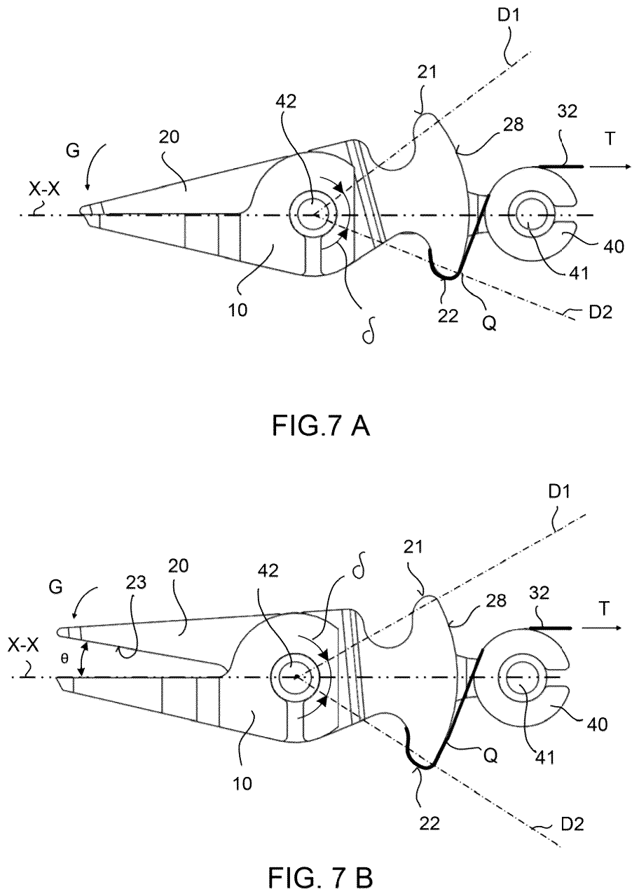

In accordance with a preferred embodiment, the radius R2 of the pulley portion 28 of the second link 20 is greater, and preferably much greater, than the radius of the sliding hub 40, as well as the radius of the winding pulley 18 of the attachment root 13 of the first link 10. As mentioned above and illustrated for example in FIG. 7, the radius R1 of the winding pulley 18 of the attachment root 13 of the first link 10 can be equal to the radius of the sliding hub 40. It is thus possible to keep the actuation tendons in the desired arrangement on the sliding surfaces 52, 53 of the support structure, where provided.

When in operation, in order to move the second link 20 with respect to the first link 10, the antagonistic actuation tendons 31, 32 slide on respective opposite sides L1, L2 of the sliding hub 40, then cross the path thereof at the cross 30 between the hub 40 and the link 20, then wind in opposite directions without sliding on the pulley portion 28 of the second link 20 at which they have ended, applying a driving action to the second link 20 which utilizes a lever arm equal to the radius R2 to deliver an increased closing (and opening) torque which is constant, i.e., it does not vary for any orientation of the second link 20 with respect to the first link 10.

By virtue of such a pulley portion 28 with a circumferential profile centered on the rotation axis G-G, the detachment of the actuation tendon 31, 32 always occurs along the same orientation (tangent to the circumference of radius R2), regardless of the opening angle of the degree of freedom of opening/closing G.

As mentioned above, in accordance with a preferred embodiment, the pulley portion 28 with a circumferential profile centered on the rotation axis G-G is flanked by opposite return surfaces 21, 22, which are not with a circumferential profile centered on the rotation axis G-G, whereby an angle & subtended by the chord (i.e., the pulley portion) can be defined, which is diagrammatically shown in FIGS. 7 A and 7 B, in which the dashed lines D1 and D2 join the rotation axis G-G with the ends A, B of the chord AB of the circumferential profile centered on the rotation axis G-G of the pulley portion 28.

Thus, a working angle can be defined as the angle formed by the local direction of the radius R2 incident at the detachment point K, Q of the tendon 31, 32 from the pulley portion 28 and a definable longitudinal extension direction, for example defined by the body of the first link 10 (the proximal portion 15 of the first link, for example) with respect to which the second link 20 is articulated.

Additionally, it is possible to deliver a closing force which is independent of the current degree (angle θ) of opening of the degree of opening/closing G. In fact, by virtue of the joint provision of such a pulley portion 28 and such a sliding hub 40 around which the actuation tendons are wound, a precise geometric relationship is determined, so that the detachment direction of the actuation tendon from the pulley portion 28 (tangent to the working radius R2) is always constant for any operating condition of the degree of freedom of opening/closing G, i.e., for any operating orientation of the second link 20 with respect to the first link 10.

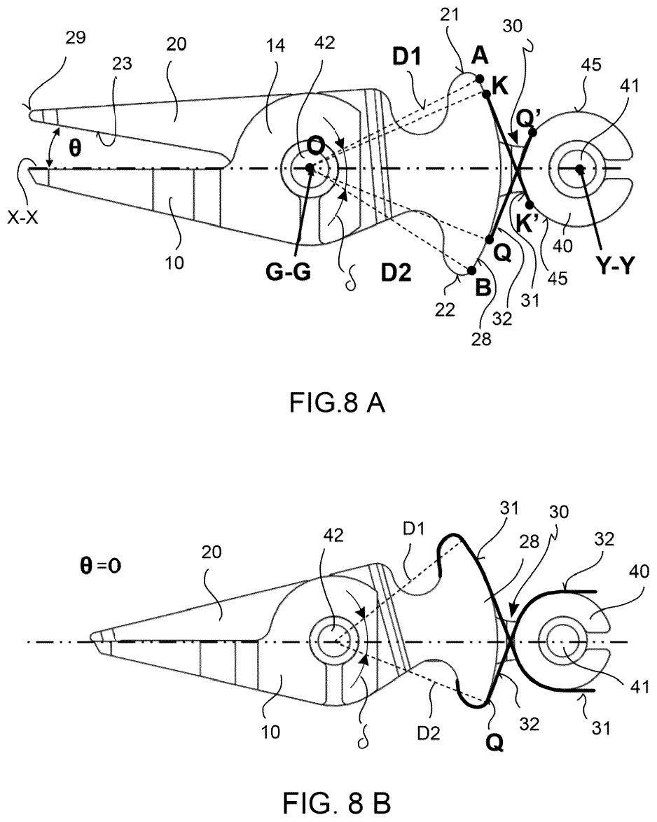

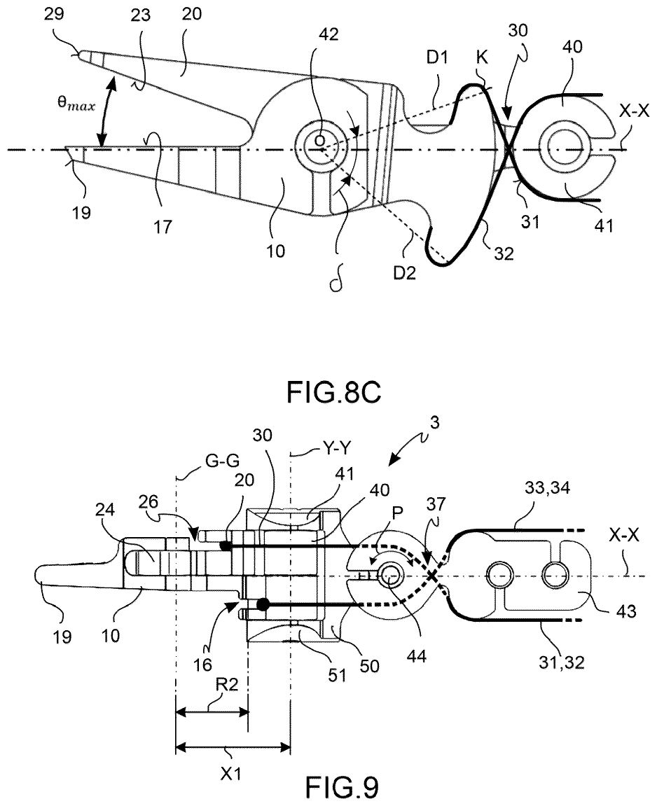

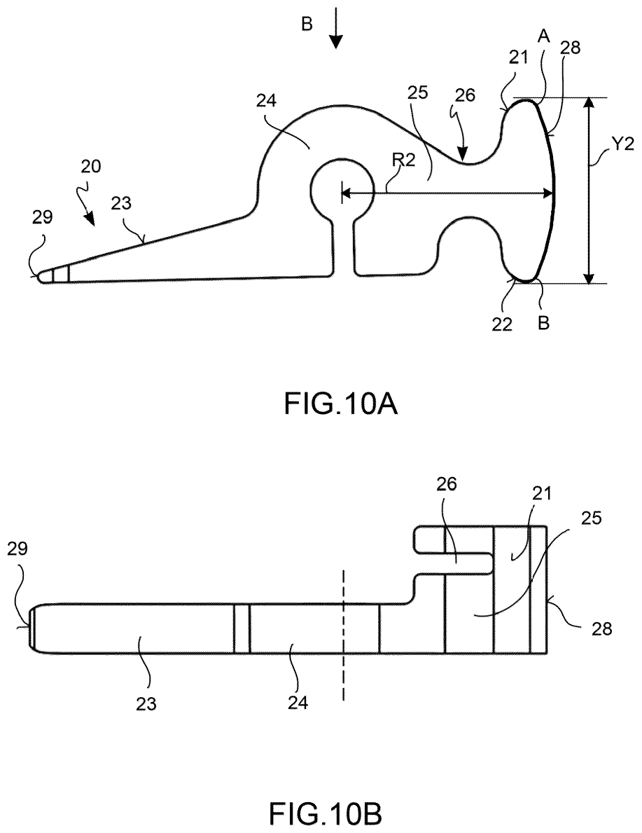

As shown for example in FIG. 8A, in which:
O indicates the rotation axis G-G,
O′ indicates the rotation axis Y-Y,
K indicates the detachment point of the actuation tendon 32 from the pulley portion 28,
Q indicates the detachment point of the actuation tendon 32 from the surface of the sliding hub 40,
K′ indicates the detachment point of the antagonistic actuation tendon 31 from the pulley portion 28,
Q′ indicates the detachment point of the actuation tendon 31 from the surface of the sliding hub 40,
A and B indicate the end points of the chord AB of the circumferential profile of the pulley portion 28,
it follows that, the distance X1 between the rotation axes Y-Y and G-G being constant, i.e., the segment O-O′ is constant, and the chord AB of the circumferential profile of the pulley portion 28 being centered in point O, then it follows that the angle K-O-O′ is of constant amplitude and equal to K′-O-O′, i.e., the segment K-Q is symmetrical to the segment P′-Q′ with respect to the direction O-O′. As a result: (i) in the case of maximum opening of the degree of freedom of opening/closing G, i.e., opening angle θ-θₘₐₓ: θ-θₘₐₓ=K′-O-B; (ii) in the case of complete closing θ=0=A-O-K; (iii) in the case of any opening θ=Q-O-B.

The tail 25 of the second link 20 having the pulley portion 28 with circumferential profile of working radius R2 centered on the rotation axis G-G can be dimensioned so that the maximum opening angle is equal to the angle subtended by the chord AB of the circumferential profile of the pulley portion 28, i.e., according to the relationship: θₘₐₓ=δ.

As shown for example in FIGS. 8B and 8C, by virtue of the provision of the pulley portion 28 having a circumferential profile centered on the rotation axis G-G, the detachment direction of the closing tendon 32 is constant (and locally orthogonal to the radius R2 of the circumferential profile of the pulley portion 28) for any opening angle θ between the links 10, 20 of the degree of freedom of opening/closing G, thus delivering a closing force which is constant for any opening/closing angle of the articulated end effector.

Preferably, the body of the first link 10 is rigid and in a single piece and forms both rotation axes Y-Y and G-G, the distance X1 is to be understood as constant for any operating condition.

As mentioned above, by virtue of such an articulated end effector 3, it is possible to obtain an increased closing force with the same traction force T transmitted by the actuation tendon.

As mentioned above, in accordance with an embodiment, said support structure of the articulated end effector comprises a support link 50 and a proximal link 43 which are articulated to each other in a rotational joint with rotation axis P-P orthogonal to the first rotation axis Y-Y, in which said rotational joint with rotation axis P-P is moved by a further pair of antagonistic actuation tendons 35, 36. The actuation tendons of the second link 20 can form a proximal cross 37 with each other between the rotation axis P-P and the rotation axis Y-Y. In accordance with a preferred embodiment, the proximal cross 37 between the actuation tendons occurs between the pair of antagonistic actuation tendons 31, 32 for moving the second link 20 and the pair of antagonistic actuation tendons 33, 34 for moving the first link 10, as shown for example in FIG. 9. In other words, the closing actuation tendon 32 of the second link 20 crosses with the actuation tendons of the first link 10 forming the proximal cross 37 and also intersects with the antagonistic actuation tendon 31 thereof (to open the second link 20) in said cross 30. Such crosses 30 and 37 are preferably longitudinally spaced and angularly offset, and for example they are offset from each other by 90°.

The support link 50 and/or the proximal link 43 can comprise one or more convex ruled sliding surfaces on which at least some, and preferably all, of the actuation tendons 31, 32, 33, 34 for moving the first link 10 and the second link 20 are adapted to slide, and in particular they slide when one of the two antagonistic tendons of the respective pair is driven (pulled).

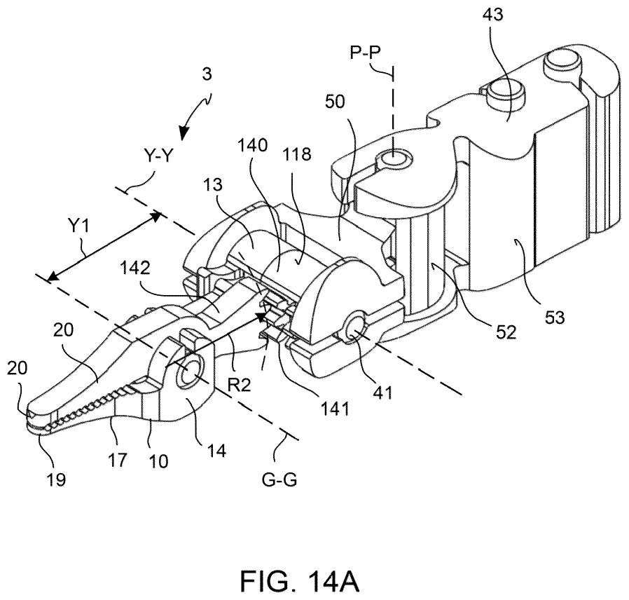

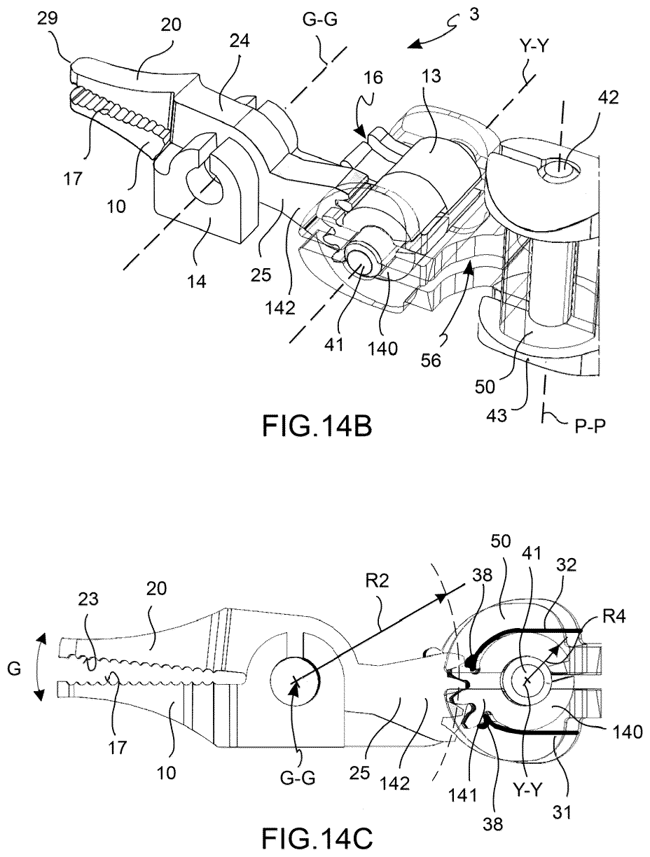

In accordance with an embodiment, as shown for example in FIG. 14A, the antagonistic actuation tendons 31, 32 of the second link 20 are both fastened to a link 140 articulated on the yaw pivot pin, which is provided as a replacement of the sliding hub 40. Therefore, in this embodiment the link 140 comprises at least one termination seat thereof for receiving the distal operating portion of the antagonistic actuation tendons 31, 32 for moving the second link 20. Accordingly, the proximal transmission tail 25 of the second link 20 can comprise, in accordance with this embodiment, transmission means of the movement action from the link 140 to the second link 20.

The circumferential actuation portion of the proximal transmission tail 25 of the second link 25 can be formed by a toothed wheel portion 142 (replacing the pulley portion 28) in operating connection with a corresponding toothed wheel counter-portion 141 provided on the toothed link 140. In such a case, the toothed wheel portion 142 defines the working radius R2 for the arm of the closing torque of the degree of freedom of opening/closing G.

In accordance with this embodiment, as shown for example in FIG. 14 C, the paths of the antagonistic actuation tendons 31, 32 for moving the second link 20, indirectly by means of the toothed wheel coupling, do not form the cross 30 between the rotation axes Y-Y and G-G. The at least one termination seat of the toothed link 140 (not shown) for receiving the actuation tendons 31, 32 can be positioned alongside the toothed portion 141 of the toothed link 140 or through at least some of the teeth of the toothed portion.

Preferably, the toothed link 140 further comprises a pulley portion 118 having winding radius R4 centered on the first rotation axis Y-Y, and the closing actuation tendon 32 is wound around the pulley portion 118 of the toothed link 140. The antagonistic opening actuation tendon 31 of the pair for moving the second link 20 can also be wound around the pulley portion 118 of the toothed link 140 and indirectly move the second link 20, by interposing a toothed wheel coupling.

The working radius R2 of the toothed circumferential profile 142 of the second link 20 is preferably greater than the winding radius R4 of the pulley portion 118 of the toothed link 140. The winding radius R4 of the pulley portion 118 of the toothed link 140 can be substantially equal to the winding radius R1 of the first link 10, and both the first link 10 and the toothed link 140 can be mounted on the first rotation axis Y-Y.

At least one, but also all, of the links 10, 20, 40, 43, 50, 140 of the articulated end effector 3 can be made by a manufacturing method including two cuts on mutually orthogonal planes, such as electro-erosion and/or laser cutting.

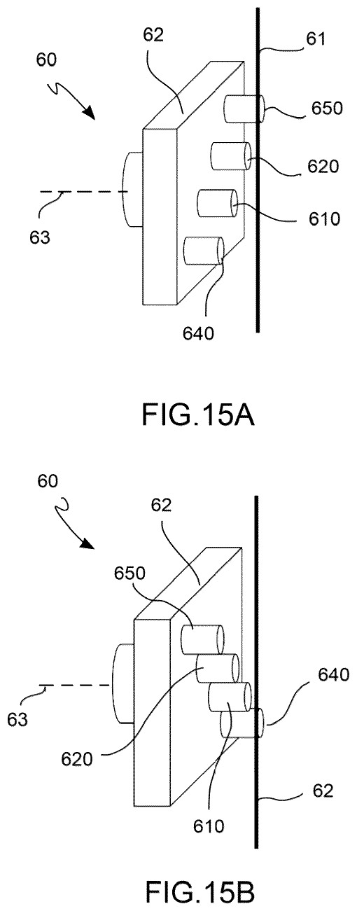

As mentioned above, at least one, but also all, of the links 10, 20, 40, 43, 50, 140 of the articulated end effector 3 can be made by a wire electro-erosion manufacturing method (WEDM) which includes making two cuts on planes orthogonal to each other with the cutting wire 61. All the links of the articulated end effector 3 can be made with the same passes of the cutting wire 61. To this end, the workpieces 610, 620, 642, 650 can be mounted on the wire electro-erosion machine 60 with a rotating tooling 62, in which they are arranged aligned along a direction (also curved) such that the cutting wire 61 intersects at most one of said workpieces 610, 620, 642, 650 at a time, when it performs both cuts on the orthogonal cutting planes, as shown for example in FIGS. 15 A-B. Preferably, replacements of the workpieces between the two cuts are avoided.

The sliding hub 40 can be made in the form of an axially perforated cylinder and therefore in accordance with a possible operating mode it is not manufactured by wire electro-erosion (WEDM). The cylindrical sliding surfaces of the sliding hub 40, or even only parts thereof, can be machined by wire electro-erosion to reduce the sliding friction with the actuation tendons 31, 32 which are intended to slide longitudinally thereon, when in operation.

The surfaces 45, 52, 53 of the links 40, 43, 50, of the articulated end effector 3 on which the antagonistic actuation tendons 31, 32; 33, 34 slide are preferably made by wire electro-erosion (WEDM). As mentioned above, the surfaces 45, 52, 53 of the links 40, 43, 50 are preferably all convex ruled surfaces with straight generators parallel to the direction of a rotation axis (axis Y-Y and/or axis P-P).

The surfaces 18, 21, 22, 28 of the links 10, 20 of the articulated end effector 3 on which the antagonistic actuation tendons 31, 32; 33, 34 wind without sliding (e.g., close to the termination seat 16, 26 thereof) are preferably made by wire electro-erosion (WEDM).

The shapes of the links 10, 20, 43, 50 can be chosen and optimized for making them by wire electro-erosion with cuts made on two orthogonal cutting planes.

By virtue of the features described above, provided either separately or in combination, where applicable, it is possible to respond to the needs mentioned above, and to obtain the listed advantages, in particular:
it is possible to decouple the axes of orientation (yaw) and opening/closing (grip), thus obtaining space for housing the working actuation radius of the second link defining the degree of freedom of opening/closing of the articulated end effector;
it is possible to obtain an increased closing force because increasing the working actuation radius of the second link defining the degree of freedom of opening/closing is allowed, with the same transverse volume of the articulated end effector.

In order to meet specific, contingent needs, those skilled in the art may make several changes and adaptations to the above-described embodiments and may replace elements with others which are functionally equivalent, without departing from the scope of the appended claims.

LIST OF REFERENCE SIGNS

| | |
|---|---|
| 1 | Surgical instrument |
| 2 | Positioning shaft or rod |
| 3 | Articulated end, or articulated end effector device, or |
| | articulated end effector |
| 4 | Transmission interface portion, or backend, of the instrument |
| 5 | Robotic system for surgical teleoperation |
| 6 | Robotic manipulator |
| 7 | Master console |
| 8 | Master control device |
| 9 | Screen |
| 10 | First link of the articulated end effector |
| 13 | Proximal attachment root |
| 14 | Connecting portion of the first link, or first connecting portion |
| 15 | Proximal section or proximal portion of the first link |
| 16 | Termination seat of the first link |
| 17 | Operating portion of the first link |
| 18 | Winding pulley portion of the attachment root of the first link |
| 19 | Distal free end of the first link |
| 20 | Second link of the articulated end effector |
| 21 | Return surface of the second link |
| 22 | Return surface of the second link |
| 23 | Distal operating portion of the second link |
| 24 | Connecting portion of the second link, or second connecting |
| | portion |
| 25 | Proximal transmission tail of the second link |
| 26 | Termination seat of the second link |
| 28 | Pulley portion with circumferential profile of the second link |
| 29 | Distal free end of the second link |
| 30 | Cross between antagonistic tendons for moving the second link |
| 31 | Actuation tendon for moving the second link to open |
| 32 | Actuation tendon for moving the second link to close |
| 33 | Actuation tendon for moving the first link |
| 34 | Antagonistic actuation tendon for moving the first link |
| 35 | Actuation tendon for moving the support link |
| 36 | Antagonistic actuation tendon for moving the support link |
| 37 | Further proximal cross of the antagonistic tendons |
| 38 | Distal termination end of the actuation tendon |
| 40 | Sliding hub or sliding bushing |
| 41 | Yaw pivot pin |
| 42 | Grip pivot pin |
| 43 | Further proximal link, e.g., link fastened to the shaft |
| 44 | Pitch pivot pin |
| 45 | Convex ruled sliding surface of the sliding hub |
| 50 | Proximal link, or support link |
| 51 | Prong |
| 52 | Convex ruled sliding surface of the support link |
| 53 | Convex ruled sliding surface of the proximal link |
| 56 | Termination seat for the actuation tendons of the support link |
| 60 | Wire electro-erosion machine |
| 61 | Cutting wire |
| 62 | Rotating tooling |
| 63 | Rotation axis of the tooling |
| 118 | Pulley portion of the toothed link |
| 123 | Longitudinal extension direction of the operating portion of the |
| | second link |
| 125 | Longitudinal extension direction of the tail of the second link |
| 140 | Toothed link |
| 141 | Toothed wheel counter-portion of the toothed link |
| 142 | Toothed wheel portion of the second link |
| 610 | Workpiece to form the first link |
| 620 | Workpiece to form the second link |
| 642 | Workpiece to form a link |
| 650 | Workpiece to form a link |
| Y-Y | First rotation axis, or yaw rotation axis |
| G-G | Second rotation axis, or opening/closing rotation axis |
| P-P | Pitch rotation axis or proximal rotation axis |
| L1 | First side |
| L2 | Second side |
| β | Angle |
| δ | Angle subtended by the pulley portion |
| R1 | Winding radius of the first link |
| R2 | Working radius of the second link |
| R4 | Winding radius of the toothed link |
| X1 | Longitudinal distance |
| X-X | Longitudinal direction |
| Y | Degree of freedom of yaw |
| G | Degree of freedom of opening/closing |
| P | Degree of freedom of pitch |
| K | Detachment point of the actuation tendon from the pulley portion |
| Q | Detachment point of the antagonistic actuation tendon from the |
| | pulley portion |
| K′ | Detachment point of the actuation tendon from the sliding hub |
| Q′ | Detachment point of the antagonistic actuation tendon from |
| | the sliding hub |
| O | Point identifying the opening/closing rotation axis |
| O′ | Point identifying the yaw rotation axis |
| A | End point of the chord of the pulley portion |
| B | End point of the chord of the pulley portion |

1. A surgical instrument comprising an articulated end effector comprising:
a support structure,
a first link articulated to the support structure, wherein the first link and the support structure are rotatable about a first rotation axis,
a second link articulated to the first link, wherein the second link and the first link are rotatable about a second rotation axis jointly defining a degree of freedom of opening/closing of the articulated end effector;
wherein:
the surgical instrument comprises a yaw actuation tendon for moving the first link with respect to the support structure,
the first link comprises an attachment root having a winding pulley portion with a radius centered on the first rotation axis,
the yaw actuation tendon for moving the first link is wound around said winding pulley portion of the attachment root of the first link; and
wherein:
the surgical instrument further comprises a closing actuation tendon for moving the second link with respect to the first link in a closing direction of the degree of freedom of opening/closing,
the second link comprises a circumferential actuation portion having a working radius centered on the second rotation axis;
the first rotation axis and the second rotation axis are separated and disjoined from each other;
the working radius of the circumferential actuation portion of the second link is greater than the radius of the winding pulley portion of the attachment root of the first link.

2. The surgical instrument according to claim 1, wherein the circumferential actuation portion of the second link is a pulley portion having a circumferential profile with a working radius centered on the second rotation axis, and wherein the closing actuation tendon for moving the second link is wound around said pulley portion of the second link.

3. The surgical instrument according to claim 2, wherein the second link comprises a body having a proximal tail, extending proximally to the second rotation axis, and wherein said proximal tail comprises said pulley portion with the circumferential profile centered on the second rotation axis.

4. The surgical instrument according to claim 3, wherein the proximal tail comprises, flanking the pulley portion, at least one return surface for returning the closing actuation tendon to move the second link.

5. The surgical instrument according to claim 1, comprising a pair of antagonistic actuation tendons, comprising said closing actuation tendon, for moving the second link with respect to the first link with antagonistic effects, wherein both actuation tendons of the pair are wound around the pulley portion with a circumferential profile of the second link, and wherein between the first rotation axis and the second rotation axis, the respective paths of the antagonistic actuation tendons for moving the second link forms a cross.

6. The surgical instrument according to claim 1, wherein the articulated end effector further comprises a sliding hub arranged proximally to the second link, and wherein the closing actuation tendon for moving the second link is wound around the sliding hub in longitudinal sliding contact.

7. The surgical instrument according to claim 1, wherein the first rotation axis and the second rotation axis are parallel and spaced apart.

8. The surgical instrument according to claim 1, wherein the working radius of the pulley portion of the second link is greater than a transverse size thereof; and/or
wherein the working radius of the pulley portion is greater than a radius of a positioning shaft of the surgical instrument.

9. The surgical instrument according to claim 1, wherein:
the circumferential actuation portion of the second link comprises a toothed circumferential profile having a working radius centered on the second rotation axis,
the articulated end effector comprises a toothed link mounted on the first rotation axis,
the toothed link comprises a circumferential toothed counter-portion centered on the first rotation axis and coupled to the toothed portion of the toothed link;
the toothed link further comprises a pulley portion with a winding radius centered on the first rotation axis,
the closing actuation tendon is wound around the pulley portion of the toothed link.

10. The surgical instrument according to claim 9, wherein the working radius of the toothed circumferential profile of the second link is greater than the winding radius of the pulley portion of the toothed link.

11. The surgical instrument according to claim 1, wherein the first rotation axis and the second rotation axis are parallel and spaced apart by a distance which is constant in every operating configuration of the articulated end effector.
